(19) United States
(12) Patent Application Publication
(10) Pub. No.: US 2015/0087981 A1
(43) Pub. Date: Mar. 26, 2015

(54) ULTRASOUND DIAGNOSIS APPARATUS, COMPUTER PROGRAM PRODUCT, AND CONTROL METHOD

(71) Applicants: Kabushiki Kaisha Toshiba, Minato-ku (JP); Toshiba Medical Systems Corporation, Otawara-shi (JP)

(72) Inventors: Hideaki ISHII, Nasushiobara (JP); Satoshi WAKAI, Nasushiobara (JP); Kensuke SHINODA, Otawara (JP)

(73) Assignees: Kabushiki Kaisha Toshiba, Minato-ku (JP); Toshiba Medical Systems Corporation, Otawara-shi (JP)

(21) Appl. No.: 14/560,810

(22) Filed: Dec. 4, 2014

Related U.S. Application Data
(63) Continuation of application No. PCT/JP2013/065879, filed on Jun. 7, 2013.

(30) Foreign Application Priority Data
Jun. 15, 2012 (JP) 2012-136412
Jun. 7, 2013 (JP) 2013-120986

(51) Int. Cl. A61B 8/08; A61B 8/00; G06T 15/08; A61B 8/14

(52) U.S. Cl. CPC A61B 8/5207; A61B 8/14; A61B 8/5269; A61B 8/466; A61B 8/54; G06T 15/08; A61B 8/463; A61B 8/523
USPC 600/440; 600/443

(57) ABSTRACT

An ultrasound diagnosis apparatus configured to generate fly-through image data on the basis of various sub-volume data that are acquired by transmitting/receiving ultrasound to/from a three-dimensional region in a subject include: a misregistration corrector configured to correct misregistration between the sub-volume data on the basis of at least any one of information on a lumen wall of a hollow organ and information on a core line that indicates the center axis of the hollow organ in the sub-volume data; a fly-through image data generator configured to generate the fly-through image data on the basis of sub-volume data where the misregistration has been corrected; and a display unit configured to display the fly-through image data.

### CROSS-REFERENCE TO RELATED APPLICATIONS

This application is a continuation of PCT international application Ser. No. PCT/JP2013/065879 filed on Jun. 7, 2013 which designates the United States, incorporated herein by reference, and which claims the benefit of priority from Japanese Patent Application No. 2012-136412, filed on Jun. 15, 2012 and Japanese Patent Application No. 2013-120986, filed on Jun. 7, 2013, the entire contents of which are incorporated herein by reference.

### FIELD

Embodiments described herein relate generally to an ultrasound diagnosis apparatus that generates wide-range fly-through image data on the basis of various sub-volume data acquired from a three-dimensional region in a subject, relate to a computer program product, and relate to a control method thereof.

### BACKGROUND

An ultrasound diagnosis apparatus emits ultrasound pulses generated by transducer elements incorporated in an ultrasound probe and receives, by using the transducer elements, reflected ultrasound waves resulting from the difference in acoustic impedance between different living tissues, thereby acquiring various types of biological information. Because recent ultrasound diagnosis apparatus, which can electrically control the ultrasound transmitting/receiving direction and the ultrasound convergence point by controlling the drive signals supplied to multiple transducer elements and controlling the delay of received signals acquired by the transducer elements, allow easy observations of real-time image data with a simple operation, i.e., causing the tip of the ultrasound probe to touch the body surface, the systems are widely used for morphologic diagnosis or functional diagnosis of living organs.

Particularly, in recent years, further advanced diagnosis and treatments have become available in which three-dimensional scanning is performed on a site to be diagnosed in a subject by using a method of mechanically moving an ultrasound probe that includes one-dimensionally arrayed multiple transducer elements or by using a method that uses an ultrasound probe that includes two-dimensionally arrayed multiple transducer elements and by generating three-dimensional image data or MPR image data using three-dimensional data (volume data) that is acquired during the three-dimensional scanning.

Another method has been proposed in which an observer's virtual viewpoint is set in a hollow organ in volume data obtained using three-dimensional scanning of a subject and the inner surface of the hollow organ to be observed (referred to as fly-through image data) is observed from the viewpoint as virtual endoscope data (see, for example, Japanese Laid-open Patent Publication No. 2000-185041).

In the above-described method of generating endoscopic image data on the basis of volume data acquired from the outside of the subject, invasion of the subject under examination is greatly reduced and, furthermore, using the method allows arbitrary setting of the viewpoint and viewing direction with respect to hollow organs, such as blood vessels or narrow parts of the gastrointestinal tract, into which it is difficult to insert an endoscope, thereby enabling examination with high accuracy, safety, and efficiency.

When the above-described fly-through image data is generated using an ultrasound diagnosis apparatus, the region on which volume data is acquired is limited to a region centered around the ultrasound probe. Thus, in order to generate the above-described wide-range fly-through image data, a method is employed in which various narrow-range volume data acquired in different positions by moving the ultrasound probe along the body surface (hereinafter, sub-volume data) are composited to generate wide-range volume data and, on the basis of the volume data, wide-range fly-through image data is generated.

In a conventional method for generating fly-through image data using wide-range sub-volume data that is acquired by compositing various sub-volume data, arithmetic operations, such as correlation operations, on a common region between sub-volume data adjacent in a hollow-organ running direction and acquired such that their ends overlap, are performed to detect misregistration between the sub-volume data and misregistration correction is performed on the basis of the result of the detection.

However, such misregistration detection and misregistration correction using all the image information on a common region have a problem in that, although mean misregistration between sub-volume data is reduced, sufficient misregistration correction is sometimes not performed on the particular hollow organ to be observed or its neighboring regions. In such a case, it is difficult to acquire fly-through image data regarding the lumen wall with good sequentiality.

### BRIEF DESCRIPTION OF THE DRAWINGS

FIG. 1 is a block diagram of an overall configuration of an ultrasound diagnosis apparatus according to an embodiment;

FIG. 2 is a block diagram of a specific configuration of a transmitter/receiver and a received-signal processor of the ultrasound diagnosis apparatus according to the embodiment;

FIG. 3 is a diagram depicting the relationship between the coordinate system for an ultrasound probe and the ultrasound transmitting/receiving direction according to the embodiment;

FIG. 4 is a diagram depicting a specific configuration and function of a misregistration corrector of the ultrasound diagnosis apparatus;

FIG. 5 is a diagram depicting misregistration-corrected sub-volume data that are adjacent in the core line direction of a hollow organ and a viewpoint that moves along the core line of these sub-volume data;

FIG. 6 is a graph showing the relationship between the viewpoint movement speed and the viewpoint-boundary distance that are set by a viewpoint movement controller according to the embodiment;

FIG. 7 is a block diagram of a specific configuration of a two-dimensional image data generator of the ultrasound diagnosis apparatus according to the embodiment;

FIG. 8 is a diagram depicting CPR image data that are generated for the purpose of monitoring sub-volume data acquisition according to the embodiment;

FIG. 9 is a diagram depicting a specific example of display data that are generated on a display unit according to the embodiment; and

FIG. 10 is a flowchart of a fly-through image data generating/displaying procedure according to the embodiment.

### DETAILED DESCRIPTION

According to embodiment, An ultrasound diagnosis apparatus that generates fly-through image data on the basis of various sub-volume data that are acquired by transmitting/receiving ultrasound to/from a three-dimensional region in a subject, the ultrasound diagnosis apparatus comprising, a misregistration corrector, a fly-through image data generator and a display unit. The misregistration corrector that corrects misregistration between the sub-volume data on the basis of at least any one of information on a lumen wall of a hollow organ and information on a core line that indicates the center axis of the hollow organ in the sub-volume data. The fly-through image data generator that generates the fly-through image data on the basis of sub-volume data where the misregistration has been corrected. The display unit that displays the fly-through image data.

Further, according to embodiment, An ultrasound diagnosis apparatus that generates fly-through image data on a hollow organ on the basis of various sub-volume data that are acquired by transmitting/receiving ultrasound to/from a three-dimensional region in a subject, the ultrasound diagnosis apparatus comprising, a viewpoint-boundary distance measuring unit, a viewpoint movement controller, a fly-through image data generator and a display unit. The viewpoint-boundary distance measuring unit that measures, as a viewpoint-boundary distance, a distance between a viewpoint that is set in the hollow organ in the sub-volume data and that moves in a direction in which the hollow-organ runs and a boundary between the adjacent sub-volume data. The viewpoint movement controller that controls, on the basis of the result of measuring the viewpoint-boundary distance, a speed at which the viewpoint moves. The fly-through image data generator that generates the fly-through image data by processing the sub-volume data on the basis of the viewpoint. The display unit that displays the fly-through image data.

Embodiments of the disclosure will be described with reference to the accompanying drawings.

### Embodiments

When an ultrasound diagnosis apparatus according to an embodiment described below generates fly-through image data of a hollow organ on the basis of volume data acquired from a three-dimensional region in a subject, an ultrasound probe is caused to move to acquire various sub-volume data that are adjacent in a direction in which the hollow organ runs and lumen-wall extraction and core line setting are performed on the hollow organ shown in each sub-volume data. Misregistration between the sub-volume data is corrected on the basis of the acquired information on the core line and lumen wall and the viewpoint that is set with respect to the core line of the misregistration-corrected sub-volume data is caused to move in the core line direction at a movement speed that is determined on the basis of the distance between the viewpoint and the sub-volume data boundary plane, thereby fly-through image data is generated in which discontinuity due to misregistration between sub-volume data is reduced.

In the following embodiments, fly-through image data are generated on the basis of sub-volume data acquired using an ultrasound probe that includes multiple transducer elements that are two-dimensionally arrayed. The fly-through image data may be generated on the basis of sub-volume data that are acquired by mechanically moving or rotating an ultrasound probe that includes one-dimensionally arrayed multiple transducer elements.

### Configuration and Functions of the System

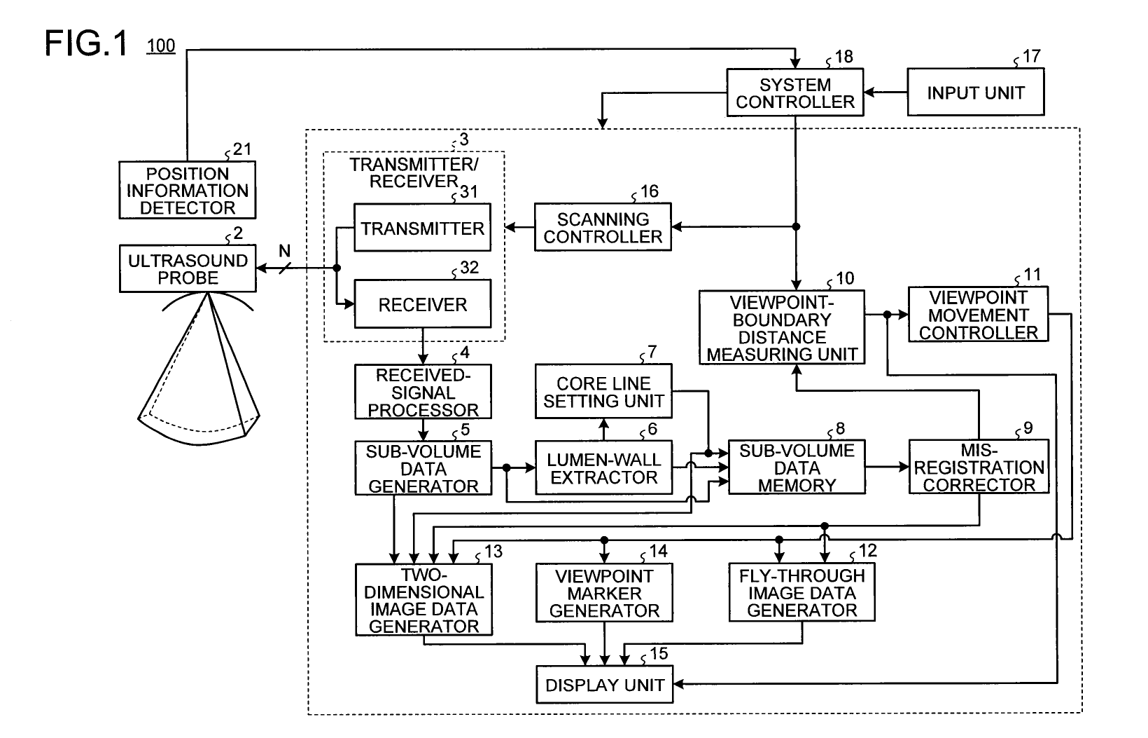

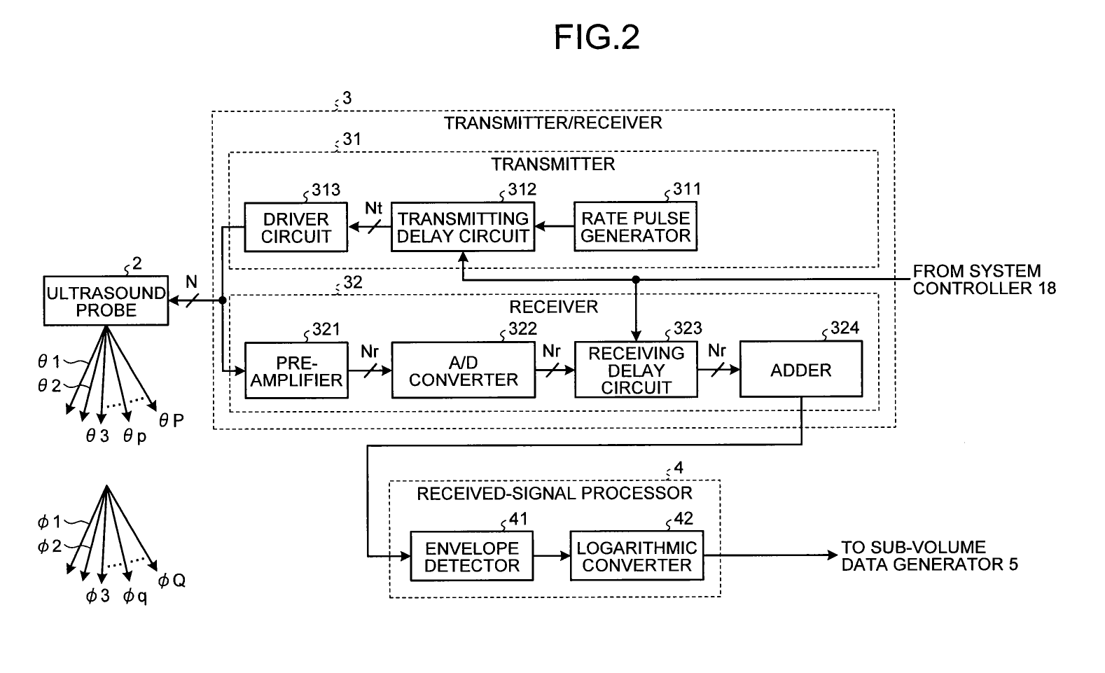

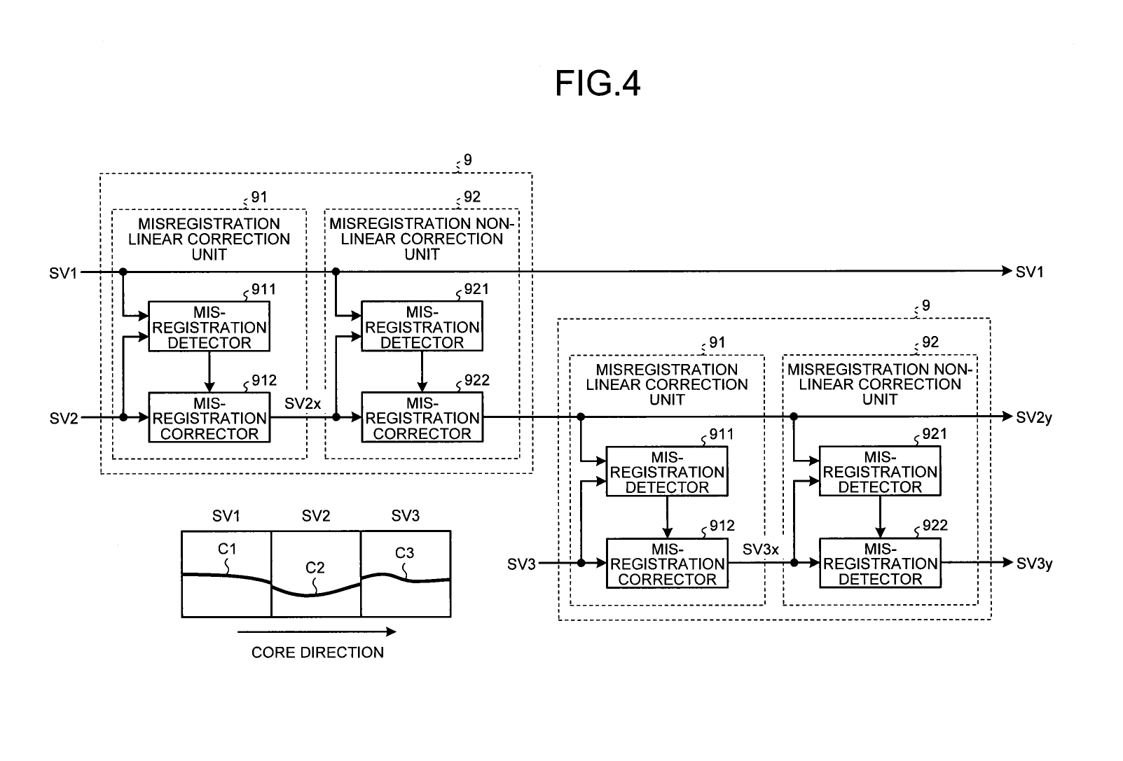

The configuration and functions of the ultrasound diagnosis apparatus according to an embodiment will be described using FIGS. 1 to 9. FIG. 1 is a block diagram of an overall configuration of the ultrasound diagnosis apparatus. FIG. 2 is a block diagram of a specific configuration of a transmitter/receiver and a received-signal processor of the ultrasound diagnosis apparatus. FIGS. 4 to 7 are block diagrams of a specific configuration of a misregistration corrector and a two-dimensional image generator of the ultrasound diagnosis apparatus.

An ultrasound diagnosis apparatus 100 of the embodiment shown in FIG. 1 includes an ultrasound probe 2 that includes multiple transducer elements that emit transmission ultrasound (ultrasound pulses) to a three-dimensional region of a subject and that electrically convert received ultrasound (reflected ultrasound waves) originating from the transmission ultrasound and acquired from the three-dimensional region; a transmitter/receiver 3 that supplies, to the transducer elements, a drive signal for emitting transmission ultrasound in a given direction of the three-dimensional region and that performs phasing and adding operations on signals that are received via multiple channels and acquired from the transducer elements; a received-signal processor 4 that processes the received signals after the phasing and adding operations; and a sub-volume data generator 5 that generates narrow-range three-dimensional image information on the basis of the B-mode data acquired per ultrasound transmitting/receiving direction (hereinafter, “sub-volume data”); a lumen-wall extractor 6 that extracts, as the lumen wall, at least the outer wall or the inner wall of the hollow organ contained in the sub-volume data; a core line setting unit 7 that sets the center axis (hereinafter, “core line”) of the hollow organ in the sub-volume data on the basis of information on the position of the acquired lumen wall; and a sub-volume data memory 8 that stores the sub-volume data to which positional information on the core line and the lumen-wall is attached.

The ultrasound diagnosis apparatus 100 further includes a misregistration corrector 9 that corrects, on the basis of the positional information on the core line and the lumen wall, misregistration between sub-volume data that are read from the sub-volume data memory 8 and that are adjacent in the direction corresponding to a direction in which the hollow organ runs (hereinafter, “core line direction”); a viewpoint-boundary distance measuring unit 10 that measures the distance between a viewpoint that moves in the core line direction along the core line and a boundary plane between the sub-volume data; a viewpoint movement controller 11 that controls movement of the viewpoint along the core line; a fly-through image data generator 12 that generates fly-through image data on the basis of the misregistration-corrected sub-volume data; a two-dimensional image data generator 13 that generates, on the basis of the sub-volume data, two-dimensional MPR (multi planar reconstruction) image data and CPR (curved multi planar reconstruction) image data; a viewpoint marker generator 14 that generates a viewpoint marker for indicating the viewpoint position in the MPR image data; and a display unit 15 that displays display data generated using the fly-through image data and the MPR image data to which the viewpoint marker is attached. The ultrasound diagnosis apparatus 100 further includes a scanning controller 16 that controls the direction in which ultrasound is transmitted to/received from the three-dimensional region of the subject; and an input unit 17 that inputs subject information, sets sub-volume data generation conditions, sets fly-through image data generation conditions, and inputs various instruction signals; and a system controller 18 that has overall control of the units.

The ultrasound probe 2 includes two-dimensionally arrayed N (N=N1×N2) transducer elements on its tip and the tip is caused to touch the body surface of the subject in order to transmit and receive ultrasound. Each of the transducer elements is connected to the transmitter/receiver 3 via a multi-core line cable for N channels (not shown). These transducer elements are electro-acoustic transducers and have a function of, when ultrasound is transmitted, converting a drive signal (electric pulse) to transmission ultrasound (an ultrasound pulse) and of, when ultrasound is received, converting the received ultrasound (reflected ultrasound waves) to a received electric signal. A positional information detector 21 that detects the position and direction of the ultrasound probe 2 is provided in or around the ultrasound probe 2.

On the basis of position signals supplied from multiple position sensors (not shown) arranged in the ultrasound probe 2, the positional information detector 21 detects positional information (position and direction) on the ultrasound probe 2 placed on the subject's body surface.

Various methods have been already proposed as methods for detecting the positional information on the ultrasound probe 2. When the detection accuracy, costs, and size are considered, it is preferable to use a method that uses an ultrasound sensor or a magnetic sensor as the position sensor. The positional information detector using a magnetic sensor includes, for example, as described in Japanese Laid-open Patent Publication No. 2000-5168, a transmitter (magnetic generator, not shown) that generates magnetism; multiple magnetic sensors (position sensors, not shown) that detect the magnetism; and a positional information calculator (not shown) that processes the position signals supplied from the magnetic sensors in order to calculate the positional information on the ultrasound probe 2. On the basis of the positional information on the ultrasound probe 2 that is detected by the positional information detector 21, positional information on the sub-volume data acquired using the ultrasound probe 2 can be obtained.

Ultrasound probes include probes for sector scanning, linear scanning and convex scanning. A medical professional (hereinafter, an “operator”) who operates the ultrasound diagnosis apparatus 100 can arbitrarily select a preferable ultrasound probe according to the site to be examined/treated. In the embodiment, the ultrasound probe 2 for sector scanning that includes two-dimensionally arrayed N transducer elements on its tip is used.

The transmitter/receiver 3 shown in FIG. 2 includes a transmitter 31 that supplies, to the transducer elements of the ultrasound probe 2, a drive signal for emitting transmission ultrasound in a given direction into a subject; and a receiver 32 that performs phasing and adding on signals that are received via multiple channels and acquired from the transducer elements. The transmitter 31 includes a rate pulse generator 311, a transmitting delay circuit 312, and a driver circuit 313.

By dividing a reference signal supplied from the system controller 18, the rate pulse generator 311 generates a rate pulse that determines the repetition period of the transmission ultrasound to be emitted into the body. The rate pulse generator 311 supplies the obtained rate pulse to a transmitting delay circuit 312. The transmitting delay circuit 312 consists of, for example, Nt transmitting transducer elements selected from the N transducer elements incorporated in the ultrasound probe 2 and the same number of independent delay circuits. During ultrasound transmission, the transmitting delay circuit 312 gives, to the rate pulse supplied from the rate pulse generator 311, a focus delay for focusing the transmission ultrasound to a given depth in order to acquire a narrow beam width and a deflection delay for emitting the transmission ultrasound in the ultrasound transmitting/receiving direction. The driver circuit 313 has a function for driving Nt transmitting transducer elements incorporated in the ultrasound probe 2 and generates a driver pulse having a focus delay and a deflection delay on the basis of the rate pulse supplied from the transmitting delay circuit 312.

The receiver 32 includes a preamplifier 321 for Nr channels corresponding to Nr receiving transducer elements that are selected from N transducer elements incorporated in the ultrasound probe 2, an A/D converter 322, a receiving delay circuit 323, and an adder 324. Signals received via the Nr channels that are supplied in the B mode from the receiving transducer elements via the preamplifier 321 are converted to digital signals by the A/D converter 322 and transmitted to the receiving delay circuit 323. The receiving delay circuit 323 gives, to each of the signals received via the Nr channels that are output from the A/D converter 322, a focus delay for focusing received ultrasound from a given depth and a deflection delay for setting high receiving directionality in the ultrasound transmitting/receiving direction. The adder 324 performs an additive synthesis on the signals that are received via the Nr channels and that are output from the receiving delay circuit 323. In other words, the receiving delay circuit 323 and the adder 324 perform phasing and adding on the received signals corresponding to the received ultrasound along the ultrasound transmitting/receiving direction.

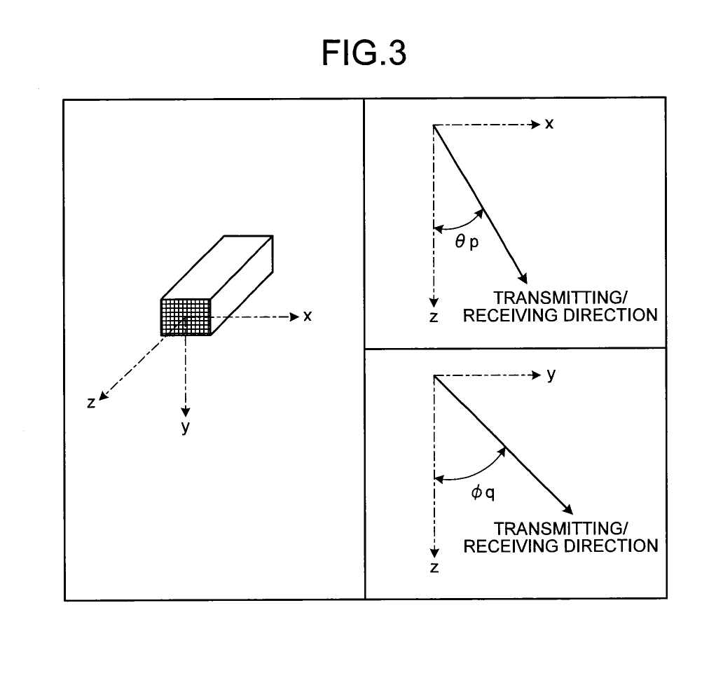

FIG. 3 depicts the ultrasound transmitting/receiving direction (θp, φq) for an orthogonal coordinate system using the center axis of the ultrasound probe 2 as the z-axis. For example, N transducer elements are arrayed two-dimensionally along the x-axis and y-axis directions. θp and φq denote transmitting/receiving direction projected on the x-z plane and y-z plane.

Here, reference is made to FIG. 2. The received-signal processor 4 includes an envelope detector 41 that performs envelope detection on each received signal that is output from the adder 324 of the receiver 32; and a logarithmic converter 42 that generates B-mode data where small signal amplitude is relatively increased by performing a logarithmic conversion operation on the envelope-detected received signals.

The sub-volume data generator 5 in FIG. 1 includes a B-mode data memory and an interpolator (not shown). Information, for example, on the transmitting/receiving direction (θp, φq) in which B-mode data of a relatively narrow-range region that is generated by the received-signal processor 4 on the basis of the received signals acquired while the ultrasound probe 2 is placed in a given position on the subject's body surface is supplied from the system controller 18 and is sequentially stored as supplementary information in the B-mode data memory.

The interpolator generates three-dimensional ultrasound data (three-dimensional B-mode data) by arraying B-mode data read from the B-mode data memory in association with the transmitting/receiving direction (θp, φq) read from the B-mode data memory and generates sub-volume data (B-mode sub-volume data) by performing interpolation, etc. on the acquired three-dimensional ultrasound data.

On the basis of the amount of a spatial change in the voxel values of the sub-volume data supplied from the interpolator of the sub-volume data generator 5, the lumen-wall extractor 6 extracts, as a lumen wall, the inner or outer wall of the hollow organ in the sub-volume data. The lumen wall of the hollow organ can, for example, be extracted by performing three-dimensional differentiation/integration operations on the voxel values of the sub-volume data and then performing a subtraction operation between the differentiated sub-volume data and integrated sub-volume data or by performing a subtraction operation between undifferentiated sub-volume data and differentiated sub-volume data. However, the method of extracting a lumen wall is not limited to the above-described one.

The core line setting unit 7 has a function of setting a core line for the lumen wall of the hollow organ that is extracted by the lumen wall extractor 6. For example, the core line setting unit 7 generates multiple unit vectors along the directions at whole three-dimensional angles using, as a reference, the base point that is pre-set in the lumen wall and selects, as a search vector, a unit vector in the direction in which the distance to the lumen wall is the maximum among the unit vectors. The core line setting unit 7 then calculates the position of the center of gravity on a lumen wall cross-section orthogonal to the search vector and newly sets, in the position of the center of gravity, a search vector whose direction is corrected such that the position where the search vector and the hollow organ cross-section coincide with the position of the center of gravity. The core line setting unit 7 repeats the above-described procedure using the corrected search vector and, during the repetition, sets the core line of the hollow organ by connecting multiple gravity center positions that are formed along the hollow-organ running direction. Note that core line setting for a hollow organ is not limited to the above-described method disclosed in Japanese Laid-open Patent Publication No. 2011-10715, etc. For example, another method such as that disclosed in Japanese Laid-open Patent Publication No. 2004-283373, etc. can be employed.

Each of the sub-volume data generated by the sub-volume data generator 5 is stored in the sub-volume data memory 8 with its supplementary information: the positional information on the lumen wall extracted by the lumen-wall extractor 6, the positional information on the core line that is set by the core line setting unit 7, and the positional information on the ultrasound probe 2 that is supplied from the positional information detector 21 of the ultrasound probe 2 via the system controller 18.

As described above, the positional information on the ultrasound probe 2 that is supplied from the positional information detector 21 and the positional information on the sub-volume data that is generated by the sub-volume data generator 5 correspond to each other. By compositing the sub-volume data in a three-dimensional space on the basis of the positional information on the ultrasound probe 2, volume data can be acquired for a wide-range three-dimensional region in the subject.

As shown in FIG. 4, the misregistration corrector 9 includes a misregistration linear correction unit 91 and a misregistration non-linear correction unit 92. The misregistration linear correction unit 91 includes a misregistration detector 911 and a misregistration corrector 912. The misregistration non-linear correction unit 92 includes a misregistration detector 921 and a misregistration corrector 922.

On the basis of the positional information on the ultrasound probe 2 (i.e., positional information on the sub-volume data), the misregistration detector 911 of the misregistration linear correction unit 91 reads two sub-volume data that are adjacent along the core line direction of the hollow organ (e.g., sub-volume data SV1 and sub-volume data SV2 shown in the lower left of FIG. 4) and the positional information on the core line C1 and the core line C2 that is added to these sub-volume data from among various sub-volume data in different imaging positions that are generated on the basis of the received signals acquired while the ultrasound probe 2 is moved along the subject's body surface and that are stored in the sub-volume data memory 8.

The regions where sub-volume data that are adjacent in the core line direction are acquired are set such that the ends of the regions overlap during observation of CPR image data that will be described below. For example, the regions where the sub-volume data SV1 and the sub-volume data SV2 are acquired are set such that a back-end neighboring region of the sub-volume data SV1 and a front-end neighboring region of the sub-volume data SV2 overlap with each other by a given amount. The back-end neighboring region and the front-end neighboring region that overlap with each other are referred to as a “back end common region” and a “front-end common region” below.

The misregistration detector 911 then calculates a cross-correlation coefficient between the positional information on the core line C2 in the front-end common region of the sub-volume data SV2 and the positional information on the core line C1 in the back end common region of the sub-volume data SV1 while translating or rotating the positional information of the core line C2. On the basis of the obtained cross-correlation coefficient, the misregistration detector 911 detects misregistration of the sub-volume data SV2 with respect to the sub-volume data SV1. On the basis of the detected misregistration, the misregistration corrector 912 of the misregistration linear correction unit 91 generates sub-volume data SV2x by performing misregistration linear correction (i.e., misregistration correction by translating or rotating the volume data SV2).

The misregistration detector 921 of the misregistration non-linear correction unit 92 detects local misregistration (distortion) of the sub-volume data SV2x with respect to the sub-volume data SV1 by performing a cross-correlation operation between the positional information on the lumen wall in the back-end common region of the sub-volume data SV1 that is read from the sub-volume data memory 8 and the positional information on the lumen wall in the front-end common region of the sub-volume data SV2x on which the misregistration linear correction has been performed by the misregistration linear correction unit 91. The misregistration corrector 922 of the misregistration non-linear correction unit 92 generates sub-volume data SV2y by performing, on the basis of the detected local misregistration, misregistration non-linear correction (i.e., misregistration correction according to a process for scaling the volume data SV2x) on the misregistration (distortion) of the sub-volume data SV2x in the vicinity of the lumen wall.

When the misregistration linear correction and the misregistration non-linear correction on the sub-volume data SV2 end, the misregistration detector 911 of the misregistration linear correction unit 91 detects, according to the same procedure, misregistration of sub-volume data SV3, which are adjacent to the sub-volume data SV2 and is read from the sub-volume data memory 8, with respect to the sub-volume data SV2. On the basis of the detected misregistration, the misregistration corrector 912 performs misregistration non-linear correction on the sub-volume data SV3 in order to generate sub-volume data SV3x.

The misregistration detector 921 of the misregistration non-linear correction unit 92 detects local misregistration (distortion) of the sub-volume data SV3x with respect to the misregistration-corrected sub-volume data SV2y. On the basis of the detected local misregistration, the misregistration corrector 922 performs misregistration non-linear correction on the misregistration (distortion) of the sub-volume data SV3x to generate sub-volume data SV3y.

According to the same procedure, misregistration linear correction and misregistration non-linear correction are performed on sub-volume data SV4, SV5, SV6 . . . (not shown) that are adjacent to the sub-volume data SV3 and the sub-volume data SV1 and the misregistration-corrected sub-volume data SV2y, SV3y, SV4y . . . are sequentially supplied to the fly-through image data generator 12.

A specific method for misregistration non-linear correction is disclosed in, for example, Japanese Laid-open Patent Publication No. 2011-024763, etc. and thus detailed descriptions of the correction method will be omitted. Regarding FIG. 4, in order to simplify the descriptions, independent units are used for misregistration correction using the sub-volume data SV1 and the sub-volume data SV2 and misregistration correction using the sub-volume data SV2 and the sub-volume data SV3. However, normally, misregistration on the sub-volume data SV2, SV3, SV4 . . . on the basis of the sub-volume data SV1 is performed by repeatedly using the misregistration corrector 9 including the misregistration linear correction unit 91 and the misregistration non-linear correction unit 92.

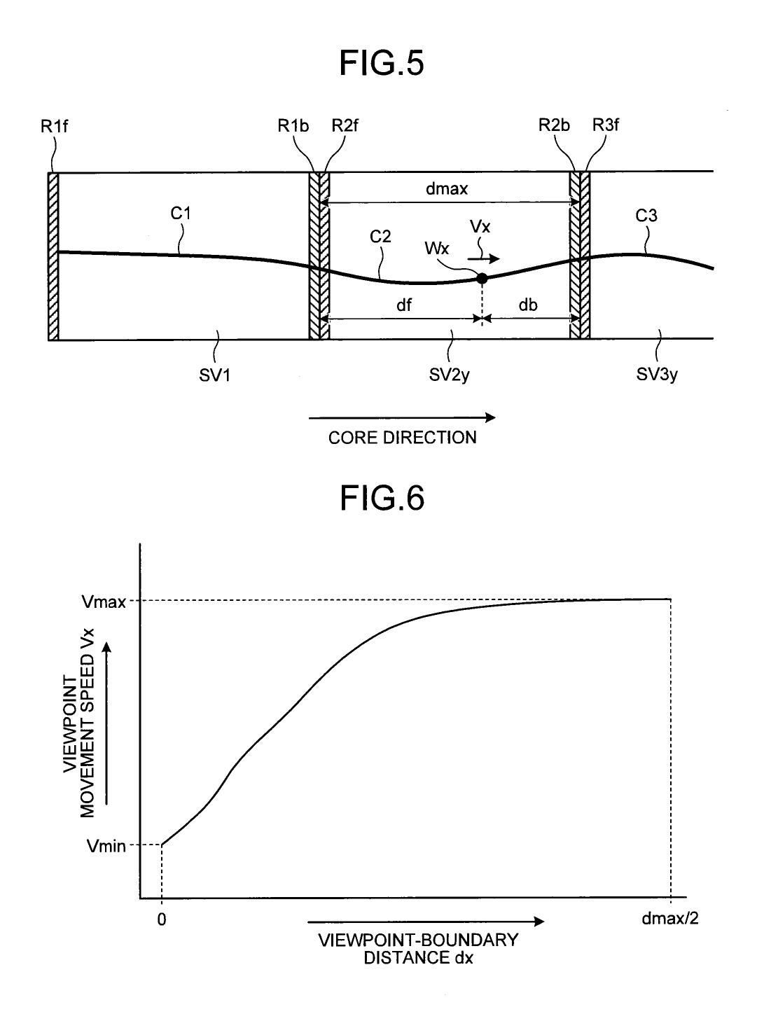

Here, reference is made to FIG. 1. The viewpoint-boundary distance measuring unit 10 has a function of measuring the distance between the viewpoint that moves in the core line direction along the core line of sub-volume data and the boundary between sub-volume data (the front end and the back end). FIG. 5 shows the sub-volume data SV1, the misregistration-corrected sub-volume data SV2y and SV3y that are adjacent to the sub-volume data SV1, and the viewpoint Wx that is initially set at the front end R1f of the sub-volume data SV1 and that moves at a given speed in the core line direction along the core line. The sub-volume data SV1, SV2y and SV3y have core lines C1 to C3 that are set by the core line setting unit 7 and on which misregistration correction has been performed by the misregistration corrector 9.

The viewpoint-boundary distance measuring unit 10 measures, as viewpoint-boundary distances, for example, a distance df from a viewpoint Wx, which moves from a front end R2f (boundary between the sub-volume data SV1 and the sub-volume data Sv2y) of the sub-volume data Sv2y to a back end R2b (the boundary between the sub-volume data SV2y and the sub-volume data SV3y), to a front end R2f and a distance db from the viewpoint Wx to the back end R2b.

Furthermore, if the viewpoint Wx continuously moves in the core line direction, the viewpoint-boundary distance is measured according to the same procedure with respect to each of the sub-volume data SV3y that are adjacent to the sub-volume data SV2y and the sub-volume data SV4y, SV5y . . . (not shown).

Here, reference is made to FIG. 1 again. The viewpoint movement controller 11 has a movement speed table (not shown) that indicates the pre-set relationship between the viewpoint-boundary distance and the viewpoint movement speed by using a lookup table, etc. The viewpoint movement controller 11 selects a viewpoint-boundary distance dx representing a small value (e.g., if df<db, dx=df) from viewpoint-boundary distances df and db that are supplied from the viewpoint-boundary distance measuring unit 10 and moves the viewpoint positioned on the core line of the sub-volume data in the core line direction at a movement speed Vx corresponding to the viewpoint-boundary distance dx that is extracted from the movement speed table.

FIG. 6 schematically shows the relationship between the viewpoint-boundary distance dx and the viewpoint movement speed Vx shown in the movement speed table. As shown in FIG. 6, the viewpoint movement speed Vx is at a maximum speed Vmax when the viewpoint Wx is at the center of the sub-volume data (dx=dmax/2) and is at a minimum speed Vmin when the viewpoint Wx is at the front end or the back end (dx=0).

The viewpoint movement controller 11 includes a viewpoint positional information calculator that calculates positional information on the viewpoint that moves along the core line and a line-of-sight direction calculator that calculates the direction of the line of sight on the basis of the positional information. The calculated viewpoint positional information and the calculated line-of-sight direction are supplied to the fly-through image data generator 12, the two-dimensional image data generator 13, and the viewpoint marker generator 14.

The fly-through image data generator 12 includes an arithmetic operator and a program store (not shown) where an arithmetic operation program for generating fly-through image data using sub-volume data is stored. On the basis of the arithmetic operation program and the viewpoint positional information and the line-of-sight direction supplied from the viewpoint movement controller 11, the arithmetic operator generates fly-through image data by rendering the registration-corrected sub-volume data supplied from the misregistration corrector 9.

When a branch is seen in the hollow organ in the fly-through image data generated by the fly-through image data generator 12 and displayed on the display unit 15, the core line direction in which the viewpoint is continuously moved is selected using an input device of the input unit 17, etc. Linear misregistration correction and misregistration non-linear correction are performed on the sub-volume data adjacent in the selected core line direction.

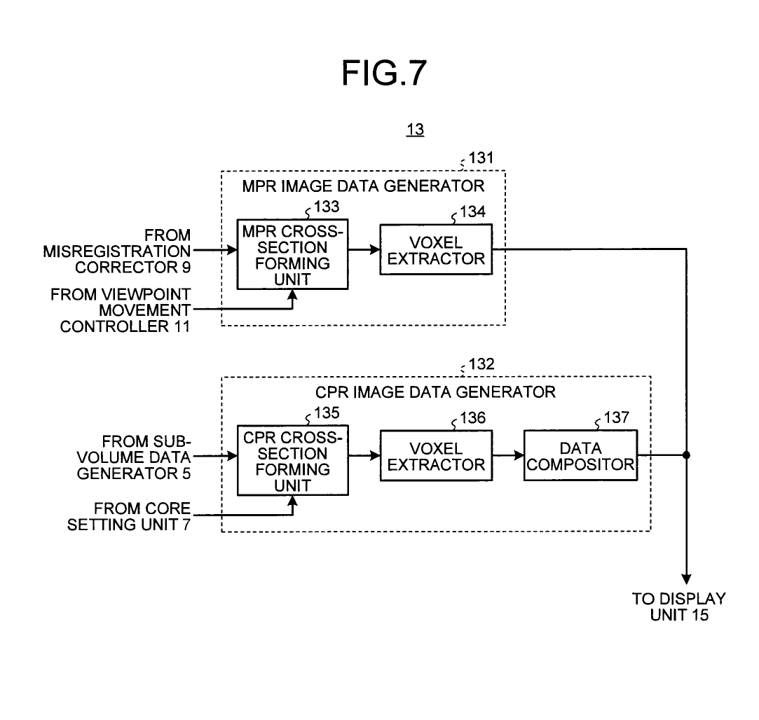

As shown in FIG. 7, the two-dimensional image data generator 13 includes an MPR image data generator 131 that includes an MPR cross-section forming unit 133 and a voxel extractor 134 and that generates MPR image data to be displayed as reference data on the display unit 15 together with the fly-through image data; and a CPR image data generator 132 that includes a CPR cross-section forming unit 135, a voxel extractor 136 and a data compositor 137 and that generates wide-range CPR image data for monitoring whether sub-volume data regarding the hollow organ of the subject is sufficiently acquired.

On the basis of the viewpoint positional information supplied from the viewpoint positional information calculator of the viewpoint movement controller 11, the MPR cross-section forming unit 133 of the MPR image data generator 131 forms three MPR (multi planar reconstruction) cross-sections (e.g., a first MPR cross-section parallel to the x-z plane, a second MPR cross-section parallel to the y-z plane and a third MPR cross-section parallel to the x-y plane in FIG. 3) that contain the viewpoint moving in the core line direction along the core line of the sub-volume data and that are orthogonal to one another. The voxel extractor 134 sets the MPR cross-section in the misregistration-corrected sub-volume data supplied from the misregistration corrector 9 and extracts voxels of the sub-volume data existing in these MRP cross-sections to generate first to third MPR image data.

The CPR cross-section forming unit 135 of the CPR image data generator 132 receives the positional information on the core line that is set by the core line setting unit 7 on the basis of the sub-volume data acquired by placing the ultrasound probe 2 in a given position and forms a CPR (curved multi planar reconstruction) cross-section having a curve plane containing the core line. The voxel extractor 136 then sets the CPR cross-section formed by the CPR cross-section forming unit 135 in the sub-volume data supplied from the sub-volume data generator 5 and projects the voxels of the sub-volume data existing in the CPR cross-section to the plane parallel to the x-y plane in FIG. 3, thereby generating narrow-range CPR image data.

The data compositor 137 composites various narrow-range CPR image data acquired by locating the ultrasound probe 2 in different positions on the subject's body surface on the basis of the positional information on the ultrasound probe 2 (i.e., positional information on the sub-volume data) added to each of the sub-volume data, thereby generating wide-range CPR image data.

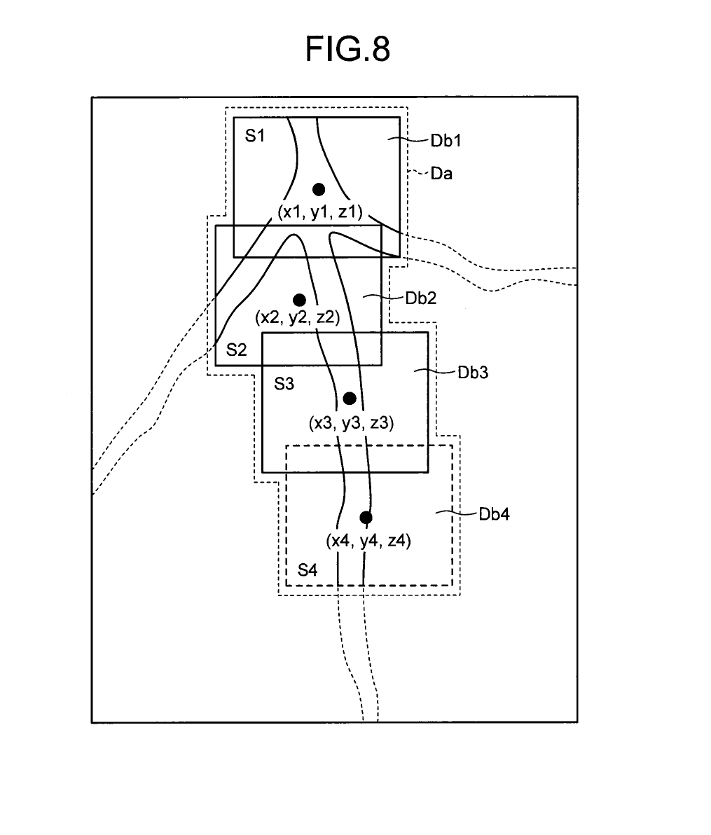

FIG. 8 shows wide-range CPR image data Da that is generated by the CPR image data generator 132. The CPR image data Da is acquired by sequentially compositing narrow-range CPR image data Db1, Db2, Db3 and Db4 based on the sub-volume data acquired by locating the center of the ultrasound probe 2 on the three-dimensional coordinates (x1, y1, z1), (x2, y2, z2), (x3, y3, z3), (x4, y4, z4) . . . on the subject's body surface.

For example, the data compositor 137 of the CPR image data generator 132 generates wide-range CPR image data Da by adding the narrow-range CPR image data Db4 of the three-dimensional region S4, which is newly acquired by moving the ultrasound probe 2 to an adjacent region, to the narrow-range CPR image data Db1 to Db3 already acquired in the three-dimensional regions S1 to S3. By adjusting the position in which the ultrasound probe 2 is placed (sub-volume data acquisition position) on the subject while observing the CPR image data Da, the operator can acquire successive sub-volume data with respect to the hollow organ. In this case, in view of the misregistration linear correction based on the core line positional information and misregistration non-linear correction based on the lumen-wall positional information, the position of the ultrasound probe 2 is adjusted such that the back-end neighboring region of the sub-volume data overlaps, by a given amount, with the front-end neighboring region of the sub-volume data that are adjacent in the core line direction.

When newly-generated narrow-range CPR image data are sequentially composited with the already-generated narrow-range CPR image data and the data is displayed on the display unit 15, the latest narrow-range CPR image data for determining a preferred position in which the ultrasound probe 2 is placed (e.g., CPR image data Db4 in FIG. 8) and other CPR image data is preferably displayed so as to be identified using a hue or brightness different from that of other CPR image data.

The viewpoint marker generator 14 in FIG. 1 has a function of generating a viewpoint marker to be added to the MPR image data generated by the MPR image data generator 131 of the two-dimensional image data generator 13 and generates a viewpoint marker of a given shape (e.g., an arrow) using supplementary information and the viewpoint positional information and the line-of-sight direction, supplied from the viewpoint movement controller 11. Usually, the shape of the viewpoint marker used is previously set for each device but it may be initially set via the input unit 17.

The display unit 15 has a function of displaying wide-range CPR image data generated by the CPR image data generator 132 of the two-dimensional image data generator 13 for the purpose of monitoring sub-volume data acquisition, the fly-through image data generated by the fly-through image data generator 12, and the MPR image data that is generated as supplementary data of the fly-through image data by the MPR image data generator 131 of the two-dimensional image data generator 13. The display unit 15 includes a display data generator, a data converter, and a monitor (not shown).

The display data generator generates first display data by converting the wide-range CPR image data (see FIG. 8) supplied from the CPR image data generator 132 into a given display format and the data converter performs conversion operations, such as D/A conversion and TV format conversion, on the above-described display data and then displays the data on the monitor.

The display data generator composites the fly-through image data supplied from the fly-through image data generator 12 and the MPR image data supplied from the MPR image data generator 131, then converts the image data into a given display format, and adds the viewpoint marker generated by the viewpoint marker generator 14 to the MPR image data, thereby generating second display data. The data converter performs conversion operations, such as D/A conversion and TV format conversion, on the display data and displays the data on the monitor. If the misregistration corrector 9 performs misregistration linear correction or misregistration non-linear correction at the boundary between the sub-volume data, the second display data to which word(s) or a symbol indicating the fact is attached may be displayed on the monitor.

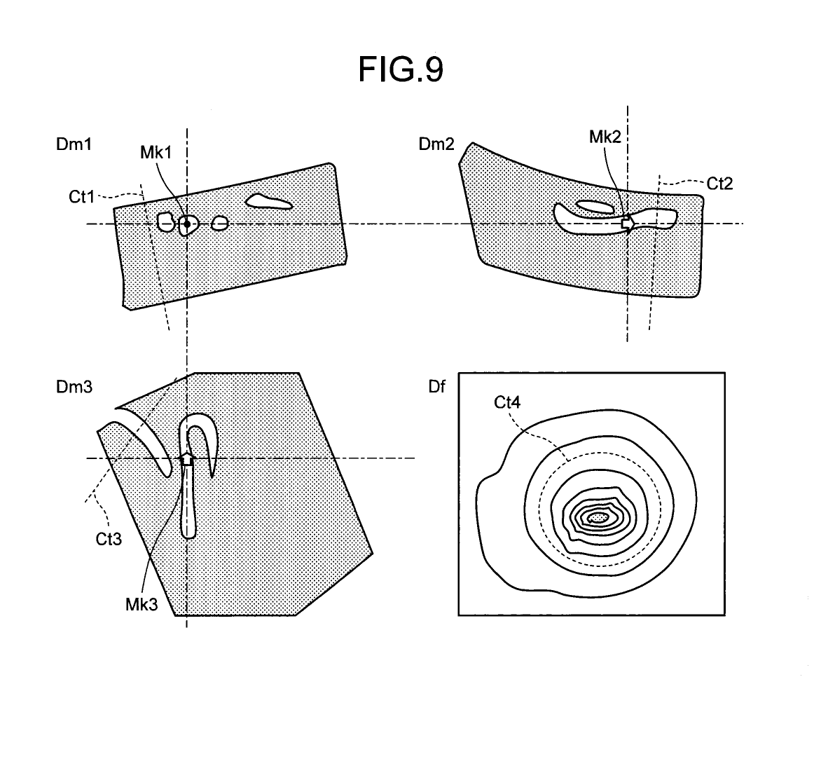

FIG. 9 shows a specific example of the second display data that are generated by the display data generator and shows first to third image data Dm1 to Dm3 on the three MPR cross-sections generated by the MPR image data generator 131, containing the viewpoint, and which are orthogonal to one another. To these MPR image data, are attached viewpoint markers Mk1 to Mk3 generated by the viewpoint marker generator 14 on the basis of the viewpoint positional information and the line-of-sight direction supplied from the viewpoint movement controller 11 and boundary lines Ct1 to Ct3 indicating the boundaries between the sub-volume data adjacent in the core line direction. In the lower right region of the second image data, fly-through image data is shown that are generated by the fly-through image data generator 12 and to which the boundary line Ct4 indicating the boundary between the sub-volume data is attached.

The MPR image data displayed together with the fly-through image data may be generated on the basis of one of the sub-volume data where there is the viewpoint or may be obtained by compositing various MPR image data generated on the basis of various adjacent sub-volume data. In this case, a boundary line indicating the boundary between the sub-volume data is added to the MPR image data and the fly-through image data, which allows accurate understanding of the positional relationship between the viewpoint that moves in the core line direction and the sub-volume data. If the viewpoint-boundary distance supplied from the viewpoint-boundary distance measuring unit 10 is shorter than a given value, i.e., if the viewpoint comes within a given distance of the boundary of the sub-volume data, the fly-through image data or a viewpoint marker may be displayed using a different hue or brightness.

The scanning controller 16 performs, on the transmitting delay circuit 312 of the transmitter 31 and the receiving delay circuit 323 of the receiver 32, delay control for performing three-dimensional ultrasound scanning for the purpose of acquiring sub-volume data in a three-dimensional region in a subject. The input unit 17 includes input devices such as a display panel on the operation panel, a keyboard, a track ball, a mouse, a selection button, and an input button. The input unit 17 inputs subject information, sets sub-volume-data generation conditions, sets MPR image data generation conditions/CPR image data generation conditions/fly-through image data generation conditions, sets image data display conditions, selects the branch in fly-through image data, and inputs various instruction signals.

The system controller 18 includes a CPU and an input information memory (not shown). Various types of information are stored in the input information memory. By generally controlling each unit of the ultrasound diagnosis apparatus 100 using the various types of information, the CPU causes each unit to acquire sub-volume data regarding the three-dimensional region of the subject, to perform misregistration correction based on the core line information or lumen-wall information of sub-volume data, and to generate fly-through image data on the basis of the misregistration-corrected sub-volume data.

### Procedure for Generating/Displaying Fly-Through Image Data

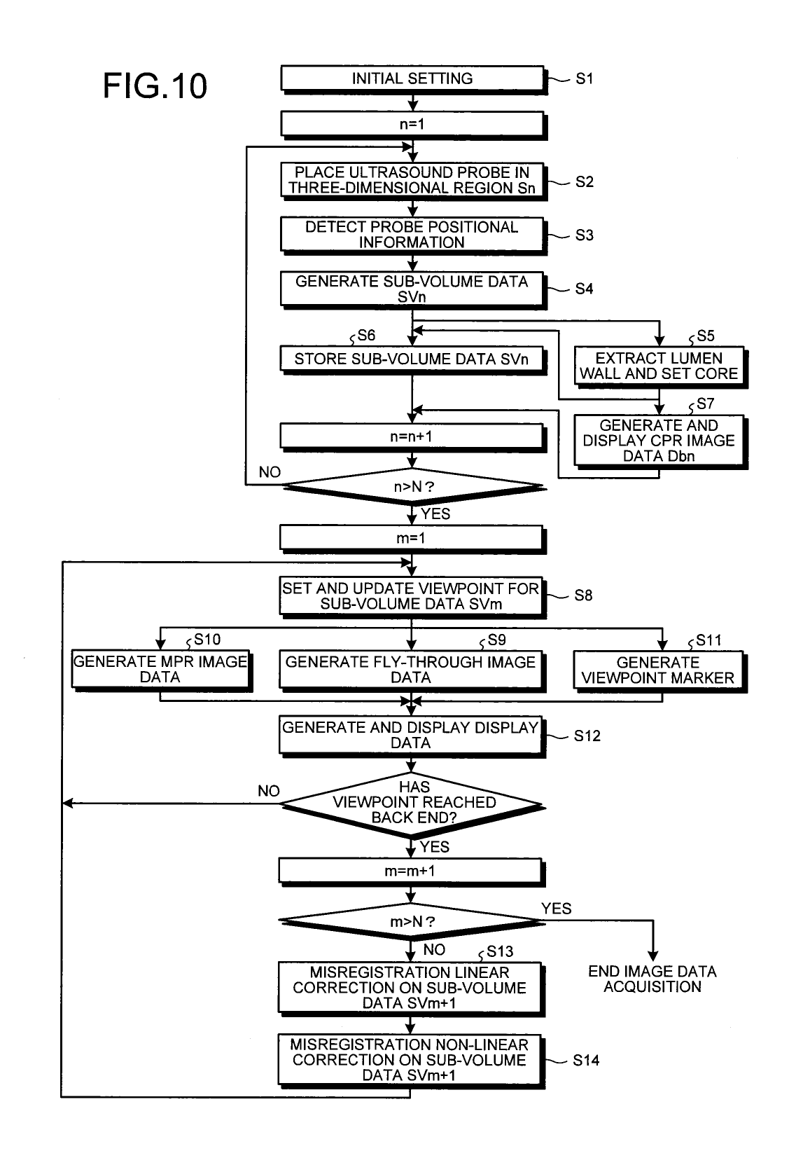

A procedure for generating/displaying fly-through image data of the embodiment will be described according to the flowchart of FIG. 10. Prior to acquisition of sub-volume data regarding the subject, the operator of the ultrasound diagnosis apparatus 100 inputs subject information using the input unit 17 and then sets sub-volume data generation conditions/MPR image data generation conditions/CPR image data generation conditions/fly-through image data generation conditions, etc. The information to be input or set by the input unit 17 is stored in the input information memory of the system controller 18 (step S1 in FIG. 10).

When the initial setting for the ultrasound diagnosis apparatus 100 ends, the operator inputs, by using the input unit 17, a sub-volume data acquisition start instruction signal while the center of the ultrasound probe 2 is placed in a position on the body surface corresponding to a three-dimensional region S1 in the subject. The supply of the instruction signal to the system controller 18 triggers acquisition of sub-volume data regarding the three-dimensional region S1 (step S2 in FIG. 10).

On the basis of the position signals supplied from the multiple position sensors provided in the ultrasound probe 2, the positional information detector 21 of the ultrasound probe 2 detects positional information (position and direction) on the ultrasound probe 2 corresponding to the three-dimensional region S1 (step S3 in FIG. 10).

When acquiring sub-volume data, the rate pulse generator 311 of the transmitter 31 supplies a rate pulse generated according to a control signal from the system controller 18 to the transmitting delay circuit 312. The transmitting delay circuit 312 gives, to the rate pulse, a delay for focusing ultrasound to a given depth in order to acquire a narrow beam width during transmission and a delay for transmitting ultrasound in the first transmitting/receiving direction (θ1, φ1) and supplies the rate pulse to the driver circuit 313 of Nt channels. On the basis of the rate pulse supplied from the transmitting delay circuit 312, the driver circuit 313 generates a driver signal having a given delay and a given shape and supplies the drive signal to Nt transmitting transducer elements, which are two-dimensionally arrayed in the ultrasound probe 2, to emit transmission ultrasound into the body of the subject.

The emitted transmission ultrasound is partly reflected from an organ boundary plane and tissues having different acoustic impedances, received by the receiving transducer elements, and converted to electric received signals of Nr channels. After the received signals undergo gain correction at the preamplifier 321 of the receiver 32 and are converted to digital signals by the A/D converter 322, the received signals are given, at the receiving delay circuit 323 for N channels, a delay for focusing the received ultrasound from a given depth and a delay for setting high receiving directionality with respect to the received ultrasound along the ultrasound transmitting/receiving direction (θ1, φ1) and undergo phasing and adding at the adder 324.

The envelope detector 41 and the logarithmic converter 42 of the received-signal processor 4 to which the received signals after phasing and adding are supplied generate B-mode data by performing envelope detection and logarithmic conversion on the received signals, and the acquired B mode data are stored in the B-mod data memory of the sub-volume data generator 5 with information on the transmitting/receiving direction (θ1, φ1) serving as supplementary information.

When generation and storing of the B-mode data in the transmitting/receiving direction (θ1, φ1) end, three-dimensional scanning is performed by performing ultrasound transmitting/receiving in the transmitting/receiving direction (θ1, φ2 to φQ), which are set by φq=φ1+(q−1)Δφ (q=2 to Q) where the ultrasound transmitting/receiving direction is updated by Δφ in the φ direction and by furthermore repeating ultrasound transmitting/receiving in φ1 to φQ in each of the transmitting/receiving directions θ2 to θP set by θp=θ1+(p−1)Δθ (p=2 to P). The B-mode data acquired by transmitting/receiving ultrasound are stored in the B mode data memory with the transmitting/receiving direction serving as supplementary information.

The interpolator of the sub-volume data generator 5 generates three-dimensional B-mode data by arraying B-mode data read from the ultrasound data memory in association with the transmitting/receiving direction (θp, φq) and furthermore generates sub-volume data SV1 by performing interpolation on the acquired three-dimensional B-mode data (step S4 in FIG. 10).

Then, on the basis of the amount of a spatial change in the voxel values of the sub-volume data SV1 supplied from the interpolator of the sub-volume data generator 5, the lumen-wall extractor 6 extracts, as a lumen wall, the inner or outer wall of a hollow organ contained in the sub-volume data SV1. The core line setting unit 7 sets the core line of a hollow organ on the basis of the positional information on the lumen wall extracted by the lumen-wall extractor 6 (step S5 in FIG. 10).

The sub-volume data SV1 of the three-dimensional region S1 generated by the sub-volume data generator 5 are stored in the sub-volume data memory 8 with supplementary information: the positional information on the lumen wall extracted by the lumen wall extractor 6, the positional information on the core line set by the core line setting unit 7, and the positional information on the ultrasound probe 2 supplied from the positional information detector 21 of the ultrasound probe 2 via the system controller 18 (step S6 in FIG. 10).

On the basis of the sub-volume data SV1 obtained in the three-dimensional region S1, the CPR cross-section forming unit 135 of the CPR image data generator 132 of the two-dimensional image data generator 13 forms a CPR cross-section of a curve plane containing the core line set by the core line setting unit 7. The voxel extractor 136 then sets a CPR cross-section in the sub-volume data SV1 supplied from the sub-volume data generator 5 and projects voxels of the sub-volume data SV1 existing in the CPR cross-section to a given plane, thereby generating narrow-range CPR image data Db1. The acquired CPR image data are displayed on the monitor of the display unit 15 (step S7 in FIG. 10).

When generating and storing of the sub-volume data SV1 with respect to the three-dimensional region S1 and generating and displaying of the CPR image data Db1 end, the operator places the ultrasound probe 2 in the position corresponding to the three-dimensional region S2 adjacent in the core line direction with reference to the CPR image data displayed on the display unit 15. By repeating steps S3 to S7, acquisition of the sub-volume data SV2 and generation of the CPR image data Db2 with respect tot the three-dimensional region S2 are performed. The CPR image data Db2 thus acquired are composited with the already acquired CPR image data Db1 and the data are displayed on the display unit 15.

By repeating the same procedure, until acquisition of sub-volume data for a three-dimensional region in a given range ends, the ultrasound probe 2 is arranged on the basis of the CPR image data (i.e., three-dimensional regions S3 to SN are set), sub-volume data SV3 to SVN are generated in the three-dimensional regions S3 to SN, a lumen wall is extracted from the sub-volume data SV3 to SVN and the core line is set in the sub-volume data SV3 to SVN, the sub-volume data SV3 to SVN are stored with the positional information on the lumen wall and the core line serving as supplementary information, and the CPR image data Db3 to DbN are generated, composited and displayed in the three-dimensional regions S3 to SN (steps S2 to S7 in FIG. 10).

When generating and storing of the sub-volume data SV1 to SVN necessary to generate fly-through image data regarding the hollow organ in the given range end, the viewpoint-boundary distance measuring unit 10 sets a viewpoint on the core line in the front end of the sub-volume data SV1 supplied from the sub-volume data memory 8 via the misregistration corrector 9 and measures the distance from the viewpoint to the back end of the sub-volume data SV1 as a viewpoint-boundary distance. The viewpoint movement controller 11 extracts, from its movement speed table, a movement speed corresponding to the result of measuring the viewpoint-boundary distance that is supplied from the viewpoint-boundary distance measuring unit 10 and moves the viewpoint set in the front end of the sub-volume data SV1 in the core line direction according to the movement speed (step S8 in FIG. 10).

On the basis of the arithmetic operation program read from its program store and the viewpoint positional information and the line-of-sight direction supplied from the viewpoint movement controller 11, the arithmetic operator of the fly-through image data generator 12 generates fly-through image data by rendering the sub-volume data SV1 supplied from the sub-volume data memory 8 via the misregistration corrector 9 (step S9 in FIG. 10).

By using the positional information on the viewpoint supplied from the viewpoint movement controller 11, the MPR cross-section forming unit 133 of the MPR image data generator 131 forms three MPR cross-sections containing the viewpoint moving in the core line direction along the core line of the sub-volume data SV1 and orthogonal to one another. The voxel extractor 134 sets the MPR cross-sections in the sub-volume data SV1 supplied from the sub-volume data memory 8 via the misregistration corrector 9 and extracts voxels of the sub-volume data existing in these MPR cross-sections, thereby generating the first to third MPR image data (step S10 in FIG. 10). The viewpoint marker generator 14 generates a viewpoint marker of a given shape with the viewpoint positional information and the line-of-sight direction supplied from the viewpoint movement controller 11 that serve as supplementary information (step S11 in FIG. 10).

The display data generator of the display unit 15 composites the fly-through image data supplied from the fly-through image data generator 12 and the MPR image data supplied from the MPR image data generator 131, then converts the image data into a given display format, and furthermore adds the viewpoint marker generated by the viewpoint marker generator 14 to the MPR image data, thereby generating display data. The data converter performs conversion operations, such as D/A conversion or TV format conversion, on the display data and then displays the display data on the monitor.

When a branch of the hollow organ is seen in the fly-through image data of the display data displayed on the display unit 15, the operator uses the input device of the input unit 17 to select a core line direction in which the viewpoint is continuously moved (step S12 in FIG. 10).

The procedure from step S8 to step S12 is repeated until the viewpoint moving in the core line direction reaches the back end of the sub-volume data SV1. Note that, according to the pre-set speed table, the shorter the distance between the viewpoint and the front end or back end of the sub-volume data SV1 is, the lower the movement speed of the viewpoint is.

When the viewpoint that moves in the core line direction along the core line has reached the back end of the sub-volume data SV1 the misregistration detector 911 of the misregistration linear correction unit 91 of the misregistration corrector 9 reads, on the basis of the positional information of the sub-volume data, the sub-volume data SV2 that are adjacent to the sub-volume data SV1 from various sub-volume data that are stored in the sub-volume data memory 8 with the positional information on the core line, the lumen wall and the sub-volume data serving as supplementary information. While translating the core line positional information of the sub-volume data SV2 in a given direction or rotating the core line positional information, the misregistration detector 911 calculates a cross-correlation coefficient between the core line positional information of the sub-volume data SV1 and the core line positional information of the sub-volume data SV2 and, by using the cross-correlation coefficient, detects misregistration of the sub-volume data SV2 with respect to the sub-volume data SV1. On the basis of the detected misregistration, the misregistration corrector 912 of the misregistration linear correction unit 91 performs misregistration linear correction on the misregistration of the sub-volume data SV2 to generate the sub-volume data SV2x (step S13 in FIG. 10).

The misregistration detector 921 of the misregistration non-linear correction unit 92 of the misregistration corrector 9 detects local misregistration (distortion) of the sub-volume data SV2x with respect to the sub-volume data SV1 by performing a cross-correlation operation between the lumen-wall positional information of the sub-volume data SV1 read from the sub-volume data memory 8 and the lumen-wall positional information on the sub-volume data SV2x on which the misregistration linear correction has been performed by the misregistration linear correction unit 91. The misregistration corrector 922 of the misregistration non-linear correction unit 92 generates sub-volume data SV2y by performing, on the basis of the detected local misregistration, misregistration non-linear correction on the misregistration (distortion) of the sub-volume data SV2x in the vicinity of the lumen wall (step S14 in FIG. 10).

When the viewpoint having reached the back-end neighboring region of the sub-volume data SV1 has been moved to the core line in the front-end neighboring region of the sub-volume data SV2y on which misregistration linear correction and misregistration non-linear correction have been performed (step S8 in FIG. 10), steps S9 to S12 are repeated to generate fly-through image data and MPR image data based on the viewpoint that moves in the core line direction along the core line of the sub-volume data SV2y, to generate a viewpoint marker and, furthermore, to display display data generated by compositing these data.

The same procedure is repeated to perform misregistration correction between adjacent sub-volume data by performing misregistration linear correction based on the core line positional information and misregistration non-linear correction based on the lumen-wall positional information of all sub-volume data SV3 to SVN generated at step S4, and to generate and display fly-through image data using the misregistration-corrected sub-volume data and the MPR image data (steps S8 to S14 in FIG. 10).

In the above-described embodiments, when fly-through image data of a wide-range region is generated on the basis of various sub-volume data that are adjacent in the hollow-organ running direction acquired from the three-dimensional region in the subject, discontinuity of fly-through image data resulting from misregistration between sub-volume data can be reduced.

Particularly, by correcting misregistration between adjacent sub-volume data by using the lumen-wall positional information extracted from the sub-volume data or the core line positional information set by using the lumen wall, the misregistration of the hollow organ at the boundary between the sub-volume data can be corrected accurately, thereby fly-through data with high continuity can be acquired.

When fly-through image data are generated, the movement speed of the viewpoint that moves in the core line direction along the core line of the hollow organ is set on the basis of the distance between the viewpoint and the sub-volume data boundary plane (viewpoint-boundary distance) and the viewpoint movement speed is reduced as the viewpoint-boundary distance shortens, so that apparent discontinuity of the fly-through image data displayed on the display unit can be reduced.

Furthermore, by performing misregistration linear correction based on the core line positional information and misregistration non-linear correction based on the lumen-wall positional information on the sub-volume data, highly-accurate misregistration correction can be performed also on complicated misregistration, whereby preferable fly-through image data can be obtained.

By compositing the narrow-range CPR image data that are generated on the basis of the sub-volume data while various sub-volume data and displaying the sub-volume data are acquired, sub-volume data that are continuous in the hollow-organ running direction can be sufficiently acquired.

Because the fly-through image data generated using the misregistration-corrected sub-volume data are composited with one or more sets of MPR image data, which are generated on the basis of the sub-volume data, to generate display data, a lot of beneficial image information for diagnosis can be acquired. Furthermore, because the viewpoint marker indicating the viewpoint position of the fly-through image data and the boundary line indicating the boundary between sub-volume data are attached to the fly-through image data and the MPR image data, the positional relationship between the viewpoint and the sub-volume data boundary plane can be accurately and easily understood.

The embodiment of the disclosure is described above. However, the disclosure is not limited to the above-described embodiment and can be modified to be carried out. For example, the misregistration corrector 9 of the embodiment extracts adjacent two sets of sub-volume data from various sub-volume data, which are acquired from the subject, on the basis of the positional information on the ultrasound probe 2 (positional information of the sub-volume data), and misregistration linear correction based on the core line positional information and misregistration non-linear correction based on the lumen-wall positional information are performed on these sub-volume data. Alternatively, prior to the misregistration linear correction and misregistration non-linear correction, conventional misregistration correction by using living-tissue information of sub-volume data may be performed. Adding this misregistration correction shortens the time necessary for misregistration linear correction and misregistration non-linear correction.

The case is described above where the misregistration non-linear correction is performed after the misregistration linear correction. However, the misregistration non-linear correction may precede or only one of the misregistration linear correction or misregistration non-linear correction may be performed.

In the above-described embodiment, the direction in which the hollow organ branches is selected by using the fly-through data. However, the branching direction may be selected using narrow-range or wide-range CPR image data generated by the CPR image data generator 132.

The case is described above where CPR image data are generated for the purpose of monitoring whether sub-volume data regarding the hollow organ of the subject is sufficiently acquired. Instead of the CPR image data, maximum-value projection image data, minimum-value projection image data or other two-dimensional image data, such as MPR image data, may be used. Particularly, generation of maximum-value projection image data or minimum-value projection image data on a projection plane parallel to the x-y plane in FIG. 3 leads to the same effect as that obtained with CPR image data.

In the above-described embodiment, misregistration correction on adjacent sub-volume data and generation of fly-through image data based on misregistration-corrected sub-volume data are performed approximately in parallel. Alternatively, misregistration correction on all sub-volume data may be performed first and then fly-through image data may be generated using the misregistration-corrected wide-range volume data. This method allows acquisition of temporally continuous fly-through image data even if misregistration correction requires a lot of time.

The case is described above where first display data including CPR image data and second data including fly-through image data and MPR image data are displayed on the common display unit 15. Alternatively, the data may be displayed in various display units. The case is described above where the sub-volume data generator 5 generates sub-volume data on the basis of B-mode data supplied from the received-signal processor 4. Alternatively, sub-volume data may be generated on the basis of other ultrasound data, such as color Doppler data or tissue Doppler data.

Furthermore, in the above-described embodiment, no-misregistration linear correction is performed on the sub-volume data where the core line is set. Alternatively, the core line may be set after misregistration non-linear correction is performed. In such a case, for example, the misregistration detector 921 of the misregistration non-linear correction unit 92 detects misregistration of the lumen wall according to the lumen-wall positional information of each of adjacent sub-volume data. The misregistration corrector 922 of the misregistration non-linear correction unit 92 then performs misregistration non-linear correction for the misregistration detected by the misregistration detector 921 to correct the misregistration of the lumen wall between adjacent sub-volume data. Thereafter, the core line setting unit 7 sets the core line for the hollow organ contained in the adjacent sub-volume data where misregistration of the lumen wall is corrected.

Each unit of the ultrasound diagnosis apparatus 100 of the embodiment may be embodied using, as hardware, a computer including a CPU, a RAM, a magnetic memory device, an input device, a display device, etc. For example, the system controller 18 that controls each unit of the ultrasound diagnosis apparatus 100 causes a processor, such as the CPU in the computer, to implement a given control program, thereby achieving various functions. In this case, the above-described computer program may be previously installed in the computer or stored in a computer-readable storage medium. Alternatively, a control program distributed via a network may be installed in the computer.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus that generates fly-through image data on the basis of various sub-volume data that are acquired by transmitting/receiving ultrasound to/from a three-dimensional region in a subject, the ultrasound diagnosis apparatus comprising:
a misregistration corrector that corrects misregistration between the sub-volume data on the basis of at least any one of information on a lumen wall of a hollow organ and information on a core line that indicates the center axis of the hollow organ in the sub-volume data;
a fly-through image data generator that generates the fly-through image data on the basis of sub-volume data where the misregistration has been corrected; and
a display unit that displays the fly-through image data.

2. The ultrasound diagnosis apparatus according to claim 1, wherein the misregistration corrector corrects the misregistration between the sub-volume data on the basis of at least any one of the information on the lumen wall and the information on the core line, the information being acquired from each of the sub-volume data that are adjacent in a direction in which the hollow organ runs.

3. The ultrasound diagnosis apparatus according to claim 2, further comprising a misregistration detector that detects misregistration of the core line, wherein the misregistration corrector corrects, on the basis of the result of detecting the misregistration of the core line, the misregistration by translating or rotating at least any one of the sub-volume data that are adjacent in the hollow-organ running direction.

4. The ultrasound diagnosis apparatus according to claim 2, further comprising a misregistration detector that detects local misregistration of the lumen wall, wherein the misregistration corrector corrects, on the basis of the result of detecting the misregistration of the lumen wall, the local misregistration by scaling up/down at least any one of the sub-volume data that are adjacent in the hollow-organ running direction.

5. The ultrasound diagnosis apparatus according to claim 4, further comprising a setting unit that sets the core line for the hollow organ that is contained in the adjacent sub-volume data where the local misregistration of the lumen wall has been corrected by the misregistration corrector.

6. The ultrasound diagnosis apparatus according to claim 2, further comprising:
a viewpoint-boundary distance measuring unit that measures, as a viewpoint-boundary distance, a distance between a viewpoint that is set in the hollow organ and that moves in the hollow-organ running direction and a boundary between the adjacent sub-volume data; and
a viewpoint movement controller that controls a speed at which the viewpoint moves,
wherein the viewpoint movement controller controls the viewpoint movement speed on the basis of the result of measuring the viewpoint-boundary distance.

7. An ultrasound diagnosis apparatus that generates fly-through image data on a hollow organ on the basis of various sub-volume data that are acquired by transmitting/receiving ultrasound to/from a three-dimensional region in a subject, the ultrasound diagnosis apparatus comprising:
a viewpoint-boundary distance measuring unit that measures, as a viewpoint-boundary distance, a distance between a viewpoint that is set in the hollow organ in the sub-volume data and that moves in a direction in which the hollow-organ runs and a boundary between the adjacent sub-volume data;
a viewpoint movement controller that controls, on the basis of the result of measuring the viewpoint-boundary distance, a speed at which the viewpoint moves;
a fly-through image data generator that generates the fly-through image data by processing the sub-volume data on the basis of the viewpoint; and
a display unit that displays the fly-through image data.

8. The ultrasound diagnosis apparatus according to claim 6, wherein the viewpoint movement controller reduces the speed at which the viewpoint moves in the hollow-organ running direction as the viewpoint-boundary distance shortens.

9. The ultrasound diagnosis apparatus according to claim 7, wherein the viewpoint movement controller reduces the speed at which the viewpoint moves in the hollow-organ running direction as the viewpoint-boundary distance shortens.

10. The ultrasound diagnosis apparatus according to claim 6, further comprising an MPR image data generator that generates MPR image data on one or more MPR cross-sections that contain the viewpoint that is set in the sub-volume data, wherein the display unit displays the fly-through image data, which are generated on the basis of the viewpoint, and the MPR image data, the fly-through image data and the MPR image data being composited.

11. The ultrasound diagnosis apparatus according to claim 7, further comprising an MPR image data generator that generates MPR image data on one or more MPR cross-sections that contain the viewpoint that is set in the sub-volume data, wherein the display unit displays the fly-through image data, which are generated on the basis of the viewpoint, and the MPR image data, the fly-through image data and the MPR image data being composited.

12. The ultrasound diagnosis apparatus according to claim 10, further comprising a viewpoint marker generator that generates a viewpoint marker that indicates the position of the viewpoint, wherein the display unit displays the viewpoint marker that is attached to the MPR image data in the position of the viewpoint.

13. The ultrasound diagnosis apparatus according to claim 11, further comprising a viewpoint marker generator that generates a viewpoint marker that indicates the position of the viewpoint, wherein the display unit displays the viewpoint marker that is attached to the MPR image data in the position of the viewpoint.

14. The ultrasound diagnosis apparatus according to claim 12, wherein when the viewpoint-boundary distance that is measured by the viewpoint-boundary distance measuring unit is shorter than a predetermined value, the display unit displays, by using different hue or brightness, at least any one of the fly-through image data and the viewpoint marker that is attached to the MPR image data.

15. The ultrasound diagnosis apparatus according to claim 13, wherein when the viewpoint-boundary distance that is measured by the viewpoint-boundary distance measuring unit is shorter than a predetermined value, the display unit displays, by using different hue or brightness, at least any one of the fly-through image data and the viewpoint marker that is attached to the MPR image data.

16. The ultrasound diagnosis apparatus according to claim 10, wherein the display unit displays a boundary line that indicates the boundary between the adjacent volume data, the boundary line being attached to any one of the MPR image data and the fly-through image data.

17. The ultrasound diagnosis apparatus according to claim 11, wherein the display unit displays a boundary line that indicates the boundary between the adjacent volume data, the boundary line being attached to any one of the MPR image data and the fly-through image data.

18. The ultrasound diagnosis apparatus according to claim 1, further comprising a CPR image data generator that generates narrow-range CPR image data on the basis of the sub-volume data and generates wide-range CPR image data by compositing various narrow-range CPR image data that are acquired in a direction in which the hollow organ runs, wherein a region where the sub-volume data are acquired is set on the basis of the wide-range CPR image data.

19. A computer program product including a computer-readable recording medium including instructions for generating fly-through image data on the basis of various sub-volume data that are acquired by transmitting/receiving ultrasound to/from a three-dimensional region in a subject, the instructions causing a computer to perform:
correcting misregistration between the sub-volume data on the basis of at least any one of information on a lumen wall of a hollow organ and information on a core line that indicates the center axis of the hollow organ in the sub-volume data;
generating the fly-through image data on the basis of sub-volume data where the misregistration has been corrected; and
displaying the fly-through image data.

20. A computer program product including a computer-readable recording medium including instructions for generating fly-through image data on a hollow organ on the basis of various sub-volume data that are acquired by transmitting/receiving ultrasound to/from a three-dimensional region in a subject, the instructions causing a computer to perform:
measuring, as a viewpoint-boundary distance, a distance between a viewpoint that is set in the hollow organ in the sub-volume data and that moves in a direction in which the hollow-organ runs and a boundary between the adjacent sub-volume data;
controlling, on the basis of the result of measuring the viewpoint-boundary distance, a speed at which the viewpoint moves;
generating the fly-through image data by processing the sub-volume data on the basis of the viewpoint; and
displaying the fly-through image data.

21. A control method performed by an ultrasound diagnosis apparatus configured to generate fly-through image data on the basis of various sub-volume data that are acquired by transmitting/receiving ultrasound to/from a three-dimensional region in a subject, the control method including:
correcting misregistration between the sub-volume data on the basis of at least any one of information on a lumen wall of a hollow organ and information on a core line that indicates the center axis of the hollow organ in the sub-volume data;
generating the fly-through image data on the basis of sub-volume data where the misregistration has been corrected; and
displaying the fly-through image data.

22. A control method performed by an ultrasound diagnosis apparatus configured to generate fly-through image data on a hollow organ on the basis of various sub-volume data that are acquired by transmitting/receiving ultrasound to/from a three-dimensional region in a subject, the control method including:
measuring, as a viewpoint-boundary distance, a distance between a viewpoint that is set in the hollow organ in the sub-volume data and that moves in a direction in which the hollow-organ runs and a boundary between the adjacent sub-volume data;
controlling, on the basis of the result of measuring the viewpoint-boundary distance, a speed at which the viewpoint moves;
generating the fly-through image data by processing the sub-volume data on the basis of the viewpoint; and
displaying the fly-through image data.
